(19) United States
(12) Patent Application Publication
(10) Pub. No.: US 2010/0145116 A1
(43) Pub. Date: Jun. 10, 2010

(54) Production of High-Purity Carotenoids by Fermenting Selected Bacterial Strains

(76) Inventors: Frederik Van Keulen, Milharado (PT); Ana Lúcia Carolas, Lisboa (PT); Mafalda Lopes Brito, Lisboa (PT); Bruno Sommer Ferreira, Lisboa (PT)

Correspondence Address: CERMAK KENEALY VAIDYA & NAKAJIMA LLP, 515 EAST BRADDOCK RD SUITE B, Alexandria, VA, 22314, US

(21) Appl. No.: 12/530,455

(22) PCT Filed: Mar. 8, 2007
(86) PCT No.: PCT/PT2007/000014

§ 371 Date: Oct. 16, 2009

(51) Int. Cl. C12P 23/00; C07C 403/24; C12N 1/20

(52) U.S. Cl. 585/23; 435/166; 435/252.1

(57) ABSTRACT

The present invention describes a process of production carotenoids in improved fermentation conditions of selected bacterial strains constitutively over-producing carotenoids or mutants thereof, purifying and isolating a specific crystalline carotenoid, preferably beta-carotene, for its use in the feed, food, cosmetic and pharmaceutical sectors. The present invention also describes a method for obtaining mutant strains constitutively overproducing carotenoids from naturally occurring bacterial strains, permitting the selection of mutants with high carotenoid yields and specificity towards a specific carotenoid. Additionally the invention describes the use of this method on obtained mutant strains for further improvement thereof. The present invention also describes said strains and improved conditions of fermentation for obtaining high concentrations of carotenoids and specificity towards a specific carotenoid, and further discloses purification steps, without cell disruption, for the extraction of carotenoids from the biomass.

### FIELD OF THE INVENTION

The present invention describes: (i) bacterial strains constitutively over-producing carotenoids, preferably beta-carotene, selected from natural isolates or mutants thereof; and (ii) the process of production of carotenoids, preferably beta-carotene, in improved conditions of fermentation, purification and isolation, yielding a specific crystalline carotenoid of high purity for its use in the feed, food, cosmetic and pharmaceutical sectors.

### STATE OF THE ART

Carotenoids are natural lipid-soluble pigments that are biosynthesised by plants, algae, fungi and bacteria, but not by animals, who have to obtain them from their diet. They are easily recognizable from the bright colours (yellow, orange, red or purple) that they often confer on the plants and micro-organisms and on animal organs when present in significant amounts (e.g. salmon). They have many different biological functions in the photosynthetic membranes of micro-organisms and plants such as species-specific coloration, photo-protection and light harvesting.

All carotenoids are hydrophobic molecules that contain a long, conjugated polyene chain, which determines not only the light absorption properties of carotenoids, and hence their colour, but also their photochemical properties, and therefore their light-harvesting and photoprotective functions. In particular, the photoprotective function is due to the ability of carotenoids to quench singlet oxygen and excited sensitizer pigments which are produced during photosynthesis, thus preventing the accumulation of harmful oxygen species. In addition, carotenoids have antioxidant properties under conditions other than photosynthesis, e.g. by interacting with free radicals and by inhibiting lipid peroxidation.

The provitamin A activity of some carotenoids has long been the focus of interest from nutritionists. For example beta-carotene and more than 50 other carotenoids can be converted to retinal, one of the forms of vitamin A, in mammals. Retinal is further oxidized in the cell to retinoic acid, the active cellular form of vitamin A. Because vitamin A cannot be biosynthesized de novo either in plants or in animals, carotenoids provide the only source of vitamin A for the entire animal kingdom. Besides this major importance of carotenoids in human nutrition, evidence has accumulated in the last 20 years that carotenoids play an important role in the prevention of cardiovascular diseases and various types of cancer. This protective action is thought to be associated with the activity of carotenoids as antioxidants. It is for this reason that carotenoids have attracted great interest from the feed, food, cosmetic and pharmaceutical industries, as they can be used not only as natural colorants, but also as high value dietary supplements and in chemoprotective formulations.

The increasing importance of carotenoids in the feed, food, cosmetic and pharmaceutical markets and some of their overlapping segments, such as the nutraceutical and cosmeceutical segments, has revamped efforts to produce carotenoids in useful amounts.

Carotenoids occur in higher plants, algae, fungi and bacteria, but also in animals such as birds and crustaceans. Carotenoids predominantly occur in their all-trans configuration which is the thermodynamically more stable isomer. The cis isomers also naturally occur or can be formed as a consequence of food processing, e.g. heating. For example, several different geometric isomers of beta-carotene (all-trans, 9-cis, 13-cis, and 15-cis isomeric forms) exist. It is known that all-trans-beta-carotene has the highest provitamin A capacity, when compared to its 13-cis- (53% activity) and 9-cis-b-carotene (38% activity) isomers (A. Schieber and R. Carle, 2005). The trans-cis iso-merization also affects bioavailability and antioxidant capacity of carotenoids. The major beta-carotene isomer in the circulation of humans is all-trans-beta-carotene, with small amounts of 13-cis and 9-cis beta-carotene. Circulating levels of the cis isomers of beta-carotene are not responsive to increased consumption of their isomers and evidences exist that the all-trans beta-carotene is selectively absorbed by the intestine or 9-cis beta-carotene is isomerized to all-trans beta-carotene between ingestion and appearance in plasma (K.-J. Yeum and R. M. Russell, 2002). Studies on the in vitro antioxidant capacity of beta-carotene stereoisomers have recently been performed and no significant differences were found between all-trans-beta-carotene, 9-cis-beta-carotene, 13-cis-beta-carotene, and 15-cis-beta-carotene (V. Böhm et al., 2002). A recent in vivo study shows that all-trans-beta-carotene restored the activity of hepatic enzymes like catalase, peroxidase and superoxide dismutase, which protect vital organs against oxidative stresses, e.g. caused by xenobiotics, when rats were fed with CCl₄. Lipid peroxidase activity, which increases when xenobiotics are present, was also maintained at normal physiological levels when all-trans-beta-carotene was included as dietary supplement when rats were fed with CCl₄ (K. N. C. Murthy et al., 2005).

### Current Carotenoid Production Methods

Carotenoids are present in many human foodstuffs, of both plant and animal origin, but are principally contained in fruit and vegetables. For example, beta-carotene can be found at relatively high concentration (0.1 mg to 1 mg/g of fresh product) in carrots. However, the seasonal variations in the carotenoid content and composition of plant sources are a disadvantage and the direct large-scale extraction of carotenoids from vegetables is not feasible, due to economic, environmental and logistic constraints.

Carotenoids such as beta-carotene and lycopene can be chemically synthesized, through reproducible and scalable processes. In fact, more than 85% of the commercially available beta-carotene is produced by chemical synthesis. Conventional chemical synthesis processes, however, use raw materials derived from fossil fuels that are processed trough high temperature, energy-intensive operating units using chemical catalysts and reagents. The chemical industry is increasingly recognizing the urgent need to diminish its dependence on petroleum-based raw-materials and fuels, to minimize its environmental impact while enhancing its competitiveness and increasing public confidence. The use of biotechnology to replace existing processes is expected to make many industries more efficient and environmentally friendly and contribute towards industrial sustainability. Waste will be reduced, energy consumption and greenhouse gas emissions will be lower and greater use will be made of renewable raw materials, typically agricultural materials converted first to simple sugars and then transformed into a wide range of end products via biological processes. Industrial biotechnology processes have the potential to revolutionize much of the current chemical-based manufacturing base.

Alternative natural sources of carotenoids are microalgae. For example, those from the Dunaliella genus can, under certain conditions, accumulate beta-carotene up to 14% of dry weight (140 mg/g). The microalgae are cultured in large-scale outdoor ponds, thus being influenced by environmental constraints, such as rainfall, sunlight and availability of salt water, since the production of high levels of beta-carotene accumulation require high salinity, high temperature and high light intensity. Nutrient limitation, especially nitrogen limitation, also enhances carotenoid formation.

In general, carotenogenesis is greatest under sub-optimal growth conditions when the specific growth rate is low. Microalgae exhibit low specific growth rates and process conditions allowing maximum biomass productivities are detrimental to the accumulation of beta-carotene, which typically require higher salt concentrations and increased exposure to sunlight, for example using shallower ponds between 5-10 cm deep [U.S. Pat. No. 4,199,895]. Facilities for microalgal production must be located where there is ample flat land available; there are cheap sources of high salinity brines, and also of lower salinity water for salinity control and to provide the water for making up evaporative losses (about 5% of total capacity/day); there are few cloudy days in the year and the mean daily temperature is higher than 30° C. for most of the year; rainfall is as low as possible; is located as far as possible from any source of pollution, meaning that the plant should not be near agricultural activities where pesticides or herbicides are used, nor industrial activities from which heavy metal contamination may occur (M. A. Borowitzka, 1990).

Due to these specificities, very few locations can be used worldwide for sustained and economic microalgal production of beta-carotene. The low cell densities achieved by the algae and their small cell size make harvesting difficult and costly. In fact, Dunaliella are small single cells with no protective cell wall and neutrally buoyant in a high specific gravity, high viscosity brine. Very large volumes are usually processed as a result of the fairly low cell densities obtained in large-scale cultures, which typically do not to exceed 1 g/L (M. A. Borowitzka, 1990), being concentrations as low as 0.25 g/L to 0.50 g/L often reported.

Conventional solid-liquid separation operations such as filtration and centrifugation generally shear-damage these cells, leading to oxidative loss of beta-carotene. In addition, the high-salt concentration brine makes corrosion of all metal equipment a major problem.

Stringent requirements must be met before the produced beta-carotene can be incorporated in human food. For example, European regulations only allow the use in food of microalgae-derived beta-carotene which is produced by the algae Dunaliella salina grown in large saline lakes located in Whyalla, South Australia. Even when performing sophisticated downstream purification, microalgal components remain in the final formulation, often conferring an unpleasant fishy taste to the food in which beta-carotene is used. All these reasons help explaining why the microalgal production of beta-carotene does not provide a viable alternative to the large-scale, established chemical synthesis process that currently accounts for more than 85% of the global beta-carotene market.

The basic technology for the industrial production of carotenoids by fungi is already set up, although there are only few examples of industrial production of fungal beta-carotene and lycopene.

The original process for the production of beta-carotene (A. Ciegler, 1965) was successively improved and currently beta-carotene concentrations of up to 9 g/L are reported (EP1367131 A1). The fermentation process uses submerged cultures of B. trispora with aeration and agitation. The production of carotenoids by B. trispora is dependent upon sexual mating of two compatible strains during the fermentation, which have to be independently grown in two parallel fermenters for about 48 hours prior to mating.

The induction of carotenoid biosynthesis is based on the diffusion of mating-type-specific pheromones, which are degradation products of beta-carotene. One of these products is trisporic acid, which acts as the major pheromone triggering the development of zygospores (A. D. Schmidt et al., 2005). This poses further difficulties to the process, as the proportions between each mating type must be optimized in order to achieve the desired beta-carotene accumulation, and/or degradation products of beta-carotene must be added to the culture in order to trigger beta-carotene accumulation.

Another difficulty is that the broth of B. trispora cultures becomes viscous and needs considerable energy input to keep it well mixed and at the required levels of dissolved oxygen. Further, due to the intricate mycelial morphology, high yields of beta-carotene extraction are only obtained after mycelium disruption in order to increase the surface area of contact between the mycelium and the extracting solvent. Mycelium disruption is achieved by drying and grinding, drying and disintegration or only disintegration of the biomass (EP1306444).

Given the complexity of the process, economic feasibility tends to be only achieved when the carotenoid production process is implemented in facilities already routinely mass-producing fungal strains (e.g. antibiotic production facilities). The possibility of using other fungi for the industrial production of beta-carotene has been undertaken only at the laboratory level, since the carotenoid accumulation is usually too low for profitable purposes (E. A. Iturriaga, et al., 2005). Additionally, some fungi preferentially produce carotenes at the surface of liquid or solid media, and consequently are hardly amenable to industrial scale production

Some yeast strains are also known to accumulate beta-carotene, such as Rhodotorula glutinis (1.1 mg/L) (P. Buzzini, 2004); Phaffia rhodozyma (10 mg/L) [EP0608172], but none have resulted in economically feasible processes.

Another approach to over-produce carotenoids has been to genetically engineer microbial strains. The efforts so far have not resulted in promising results. For example, transformed E. coli are typically reported to accumulate beta-carotene up to 1 mg/g of dry biomass [U.S. Pat. No. 5,656,472]. Recently, metabolically engineered E. coli strains were reported to accumulate beta carotene up to 30 mg/g dry biomass (S.-W., Kim, et al., 2006), but the purity of the thus produced beta-carotene remained at only about 80% in relation to total carotenoids produced. Several other works reported the cloning of carotenogenic genes not only in bacterial strains (besides E. coli, also Methylomonas strains were recently engineered, as reported in U.S. Pat. No. 6,929,928), but also in fungi (Fusarium sporotrichioides, U.S. Pat. No. 6,372,479; Phycomyces blakesleeanus, EP0587872; Aspergillus nidulans, U.S. Pat. No. 5,656,472) and in yeasts (Saccharomyces cerevisiae, U.S. Pat. No. 5,656,472; Pichia pastoris, U.S. Pat. No. 5,656,472). Typically, the production titres achieved are extremely low. In addition, since several genes must be cloned in order to produce carotenoids in recombinant non-carotenogenic bacteria, the recombinant strains tend not to be stable.

Carotenoids produced with a recombinant microbial strain would be difficult to bring to the market, since most carotenoids are used for human and animal nutrition, segments in which very stringent regulations keep genetically modified organisms-derived ingredients or additives out of products for human and animal nutrition. Even if such genetically modified organisms-derived ingredients or additives would be authorized, after an extremely lengthy and expensive procedure for proof of safety, they would have to be labelled as being derived from a genetically modified organism and the food or feed in which they would be incorporated would also have to be labelled as containing components derived from a genetically modified organism. Given the consumer opposition against food products derived from genetically modified sources, this is a critical drawback in the production of compounds targeted to the food and feed market, such as carotenoids, using recombinant technology.

### Naturally Occurring Bacterial Strains Producing Carotenoids

Several carotenogenic bacterial strains were reported. These include Cyanobacteria, Erwinia uredovora (Pantoea ananatis), Erwinia herbicola (Pantoea agglomerans), Flavobacterium, Rhodomicrobium vannielii, Protaminobacter ruber, halophilic bacteria and Mycobacteria.

These organisms however typically produce a mix of carotenoids at very low concentrations, remaining below 1 mg/g of dry biomass. Additionally, these organisms typically exhibit low specific growth rates and therefore low carotenoid production rates and low process productivities, thus not enabling their use in industrial, economically feasible processes. In fact, bacteria reported to date that naturally produce beta-carotene show extremely low intracellular concentrations and low specificity towards beta-carotene.

The occurrence of carotenoids in several bacterial strains is known since the end of the 19^{th} century (M. A. Ingraham and C. A. Baumann, 1933), although only later the available analytical techniques allowed a more precise identification and quantification of the pigments.

Mycobacterium kansasii was reported to accumulate beta-carotene at levels up to 75% with respect to total carotenoids, but the intracellular concentration of beta-carotene did not exceed 0.80 mg/g cell dry weight (H. L. David, 1974a; H. L. David, 1974b). Further, the growth rate of Mycobacteria is typically low and these strains tend to adhere to surfaces, even to stainless steel, which makes them extremely difficult to grow in bioreactors and further process them. Additionally, evidence exists that carotenogenesis in Mycobacteria and other strains is a photoinduced process, thus posing further limitations in large scale processing in bioreactors.

A process using Flavobacterium multivorum ATCC 55238 was recently reported [US20050214898A1] in which the concentration of beta-carotene reached 2.4 mg/g of dry biomass with maximum beta-carotene purities of 80% with respect to total carotenoids. In this method, however, salts or compounds of the tricarboxylic acid cycle had to be added at some point during growth in order to trigger carotenoid accumulation. Additionally, this strain is a Biosafety Level 2 micro-organism according to the German Collection of Microorganisms and Cell Cultures and the American Type Culture Collection, meaning that it is associated with human disease. The same happens for the Pantoea agglomerans, Mycobacterium kansasii strains referred above.

The isolation of a Sphingomonas strain which allowed the production of 16 mg/L beta-carotene, with a purity of 71% with respect to total carotenoids was reported (Silva et al., 2004). When processed in shake flask with the addition of further nutrients, production levels of 45 mg/L beta-carotene could be achieved with a purity of 89%, but only after 7 days of culture.

Recently, a method using a Paracoccus strain was presented that claimed the production of beta-carotene at 100% purity. However a concentration of only 16 mg/L culture broth was reported (EP1676925). These production and productivity levels are still insufficient and do not allow an economically feasible processes to be implemented.

Thus, to the best of our knowledge, and despite much effort put forward by several researchers and companies, there are no feasible processes available for the production of natural beta-carotene with high consistency which combines high productivity, simplicity, reproducibility, robustness and ease of scale-up and purification, using renewable and cheap raw-materials derived from agricultural products or wastes. The present invention describes a novel process using a new naturally occurring strain constitutively over-producing beta-carotene isolated from nature, with a one-step easily controllable and scalable fermentation, using cheap and renewable raw materials, yielding beta-carotene with high purity for use, for example but without limitation, in the feed, food, cosmetic and pharmaceutical sectors.

### Method for Obtaining Mutant Strains Constitutively Over-Producing Carotenoids

In this invention, naturally occurring strains identified as belonging to the Sphingomonas genus, constitutively producing beta-carotene were isolated from soil. Commonly to all carotenogenic organisms, the isolated strains produce a mix of carotenoids which occur throughout the carotenogenic pathway.

In this invention, a method for the improvement of the isolated strains was developed so that to maximize the carotenoid intracellular concentration and to maximize the purity of beta-carotene with respect to other carotenoids, without the need of chemical or physical induction or addition of building blocks used in the tricarboxylic acid cycle or the carotenogenic pathway.

The mutagenic method of the invention was adapted from classical mutagenesis techniques in such a way as to combine trials in which mutagenic conditions are set to obtain survival rates lower than 10% with trials designed in such a way as to promote spontaneous mutations of the strains and their detection.

The mutagenic method of the invention uses a series of detection methods, both based on visual colour observation of the colonies formed by the mutants and on the spectrophotometric and chromatographic analysis of the pigment composition of the mutants, especially those that exhibited a detectable colour difference with respect to their parent strain. The method of the invention thus provides a fast, easy to implement way to produce improved carotenoids over-producing strains form out-performing natural isolates.

The method of the invention allowed obtaining a mutant strain that accumulated much higher levels of beta-carotene than the original isolate and with much higher purity, as shown in Examples 1 and 2. Upon alignment of 16S rRNA gene sequence of the isolated strain and the obtained mutant, a similarity of 98% was observed.

Phylogenetic analysis suggests that the mutant strain obtained through this invention, further characterized bellow, is a novel Sphingomonas strain. The Sphingomonas mutant strain obtained in this invention has much higher growth rates when compared to other carotenogenic micro-organisms (up to 0.20 h⁻¹, allowing to attain 100 optical density units in less than 48 h), accumulates beta-carotene at levels higher than 10 mg/g of dry cell weight and naturally accumulates beta-carotene over other carotenoids, allowing obtaining a beta-carotene purity higher than 90% with respect to total carotenoids.

All these parameters are major improvements with respect to the methods for the production of beta-carotene using naturally occurring bacteria available in the state of the art and provide for the first time a production strain attractive for the large-scale commercial production of natural beta-carotene. Additionally, the selected strain is safe and amenable to large scale production, similarly to other processes using Sphingomonas strains to produce food additives, such as biotin [EP0589285] and gellan gum (Kang et al., 1982)

### Process for the Large Scale Production of Carotenoids

The invention also provides for the first time a process for the large scale culture of bacterial strains, obtained using the selection and mutagenic method of this invention, that maximizes the production of biomass, the production of carotenoids, preferably beta-carotene, per unit of biomass and the specificity of the production of a specific carotenoid, preferably beta carotene, with respect to the total carotenoids. The process of this invention uses renewable raw materials derived from agricultural products and wastes which are fed to the producing strain throughout a culture in closed and controlled bioreactors in such a way as to maximize the growth and in conditions that lead to the accumulation of a specific carotenoid, preferably beta-carotene, without the need to perform physical or chemical induction or to add any building blocks used in the tricarboxylic acid cycle or the carotenogenic pathway. The invention provides optimum ranges of culture parameters such as temperature, pH, dissolved oxygen concentration, concentration of nutrients that maximize both the biomass produced and the carotenoid accumulation and their respective productivities. As far as we know, the process of the invention is the first process described so far for the production of beta-carotene using naturally occurring bacterial strains or mutants thereof in controlled bioreactors mimicking those used at industrial scale, thus establishing the conditions to be used in a production plant.

All biological methods for the production of carotenoids available involve the rupture of the cells in which the carotenoid accumulates. As such, the carotenoids are mixed in a fraction containing a complex mixture of cellular components, making the purification steps required to obtain a carotenoid with required purity complex and expensive. The purification steps of the process of the present invention have the important advantage of allowing for the direct extraction of the substantially pure carotenoid from the biomass without any prior cell disruption steps. In this way, the purification steps of the process of the present invention do not need to include the separation of the carotenoids from the fraction of bulk cellular components released when the cell is disrupted. This not only reduces the steps needed in the downstream process, but also provides a very clean carotenoid extract by means of a simple and cost-effective extraction step.

### General Description of the Invention

The present invention describes naturally occurring bacterial strains constitutively over-producing carotenoids, particularly beta-carotene, or mutants thereof, particularly a new bacterial strain belonging to the Sphingomonas genus over-producing beta-carotene, and mutants thereof, a process using said naturally occurring bacterial strains or mutants thereof comprising a reproducible, robust, easy to control and scalable fermentation step, using cheap and renewable raw materials derived from agricultural products or wastes, that after simple purification allows to obtain high yields of carotenoids, particularly substantially pure carotenoids, most preferably substantially pure beta-carotene for use, for example but without limitation, in the feed, food, cosmetic and pharmaceutical sectors.

This invention comprises the following aspects: (i) the selection of bacterial strains constitutively over-producing carotenoids, preferably beta-carotene, obtained from natural isolates or mutants thereof; and (ii) the development of a process including improved and controlled conditions of fermentation using a naturally occurring bacterial strain or mutant thereof, aiming at maximizing the amount of biomass produced per volume and per time and at maximizing the amount of carotenoid, particularly substantially pure carotenoid, most preferably substantially pure beta-carotene, produced per unit biomass and per time, and the purification of said carotenoid, particularly substantially pure carotenoid, most preferably substantially pure beta-carotene using natural solvents.

The features of the process of the present invention makes it competitive with the chemical synthesis method presently used industrially and with the existing alternatives for the biological production of beta-carotene.

The expression ‘naturally occurring bacterial strain’ in relation to the definition of the present invention indicates any bacteria that can be isolated from any source in nature, particularly soil, which naturally and constitutively produces carotenoids.

The expression ‘substantially pure carotenoid’ in relation to the definition of the present invention indicates that the amount of a specific carotenoid produced by the naturally occurring bacterial strain is higher than 50% of total carotenoids produced by said bacterial strain, preferably higher than 80% of total carotenoids produced by said bacterial strain, most preferably higher than 90% of total carotenoids produced by said bacterial strain.

The expression ‘substantially pure beta-carotene’ in relation to the definition of the present invention indicates that the amount of a beta-carotene produced by the naturally occurring bacterial strain is higher than 50% of total carotenoids produced by said bacterial strain, preferably higher than 80% of total carotenoids produced by said bacterial strain, most preferably higher than 90% of total carotenoids produced by said bacterial strain.

The expression ‘maximizing the amount of biomass’ in relation to the definition of the present invention indicates achieving a biomass concentration of at least 20 optical density units measured at 600 nm, preferably at least 50 optical density units measured at 600 nm, most preferably at least 100 optical density units measured at 600 nm.

The expression ‘maximizing the concentration of the substantially pure carotenoid’ in relation to the definition of the present invention indicates achieving a concentration of the substantially pure carotenoid of at least 1 mg/g on the basis of cell dry weight, preferably of at least 3 mg/g on the basis of cell dry weight, most preferably of at least 5 mg/g on the basis of cell dry weight, even more preferably of at least 10 mg/g on the basis of cell dry weight.

The expression ‘natural solvent’ in relation to the definition of the present invention indicates any solvent that is toxicologically innocuous and/or is included in class III of the ICH guidelines (International Conference of Harmonization).

(i) Production and Selection of Mutants Constitutively Over-Producing Carotenoids from Naturally Occurring Bacterial Strains

The present invention provides a method for the improvement of naturally occurring bacterial strains based on the production of mutants using classical mutagenic techniques or by spontaneous mutation coupled to a series of phenotypical tests aiming at identifying strains over-producing carotenoids, preferably over-producing a substantially pure carotenoid, most preferably over-producing substantially pure beta-carotene.

In this invention, a method for the improvement of the isolated strains was developed so that to maximize the carotenoid intracellular concentration and to maximize the purity of beta-carotene with respect to other carotenoids.

The mutagenic method of the invention was designed in such a way as to combine trials in which mutagenic conditions are set to obtain survival rates lower than 10% with trials designed in such a way as to promote spontaneous mutations of the strains. Thus it is designed in such a way that it allows obtaining isolated, easy to pick individually, colonies of cells that survived the trials.

The mutagenic method of the invention uses a series of detection methods, both based on visual colour observation of the colonies formed by the mutants and on the spectrophotometric and chromatographic analysis of the pigment composition of the mutants, especially those that exhibited a detectable colour difference with respect to their parent strain.

Criteria used in the method of this invention for selecting mutant strains with improved characteristics in relation to those of their parent strain are an increase of at least 5% of accumulated total carotenoids or single carotenoid, preferably beta-carotene per unit of biomass or unit of culture liquid or an increase of at least 5% of the accumulated fraction of single carotenoid, preferably beta-carotene, in relation to total carotenoids. The procedure herein described for obtaining mutants with improved single carotenoid, preferably beta-carotene, production can be also applied to obtained mutants so as to improve their performance even further.

The mutagenic method of the invention allowed the improvement of a Sphingomonas strain isolated from soil (SEQ ID: 1) that naturally accumulated carotenoids. This isolated strain had a specific growth rate of 0.18 h⁻¹, it constitutively accumulated carotenoids at a concentration of 1.7 mg/g dry cell weight, of which 29% was beta-carotene.

After its selected progeny was submitted three times to the mutagenic and selection method of the invention, a new Sphingomonas strain was obtained, Sphingomonas sp. M63Y (SEQ ID: 2), which, when cultured in shake flasks, exhibits a high growth rate (0.20 h⁻¹) and accumulates constitutively carotenoids with high specificity towards beta-carotene, at a concentration of 4.8 mg/g dry cell weight, with a purity of 78% with respect to total carotenoids.

(ii) Production Process of High Purity Carotenoid with Improved and Controlled Conditions of Fermentation Using the Selected Over-Producing Bacterial Strains

The fermentation step of the novel process using a naturally occurring strain over-producing carotenoid, preferably beta-carotene, or a mutant thereof can be carried out in any customary way, such as batch fermentation, fed-batch fermentation, continuous fermentation, with or without cell recycle, or any combination or any variation thereof. Since the fermentation conditions that allow the highest productivity and yield in terms of biomass production are different than those that allow the highest productivity and yield in terms of production of substantially pure carotenoid, the fermentation may comprise different stages with different aims. For example, stages may exist aiming at maximizing the biomass concentrations, while other stages may exist aiming at maximizing the concentration of the substantially pure carotenoid. These stages can be combined in any appropriate order, although it is preferred that in the first stage fermentation conditions are such that maximize the biomass concentration.

A stage in which fermentation conditions are such that maximize the biomass concentration can be followed by a stage in which fermentation conditions are such that maximize the concentration of the substantially pure carotenoid or by a stage in which fermentation conditions are different from those of the previous stage but that also aim at maximizing the concentration of biomass.

A stage in which fermentation conditions are such that maximize the concentration of the substantially pure carotenoid can be followed by a stage in which fermentation conditions are such that maximize the biomass concentration or by a stage in which fermentation conditions are different from those of the previous stage but that also aim at maximizing the concentration of the substantially pure carotenoid.

Additionally, stages in which the fermentation conditions allow a compromise between biomass and carotenoid production can be included in the overall fermentation process.

The fermentation mode used during each stage can be individually chosen from the fermentation modes set forth above, such as batch fermentation, fed-batch fermentation, continuous fermentation, with or without cell recycle, or any combination or any variation thereof.

The conditions inside the bioreactor at each of said stages be can be individually set in terms of temperature time profile, pH time profile, dissolved oxygen concentration time profile, feeding rate, or any other parameter that influences the culture performance. When the fermentation mode involves, at any stage, nutrient feeding, the feeding rate can be determined a priori, for example, using a constant feed rate or using a feed rate calculated by a mathematical equation correlating the limiting nutrient requirements to the expected growth rate and the expected biomass/nutrient yield or any other predetermined suitable feeding regime readily established by anyone skilled in the art.

The nutrient feeding rate can also be triggered by any kind of control loop based on the control of, for example but without limitation to, pH, dissolved oxygen, oxygen and/or carbon dioxide concentration in the fermentation exhaust gas, respiratory quotient, glucose concentration or any other carbon source concentration, or any combination thereof.

Other additions to the fermentation, at any of its stages, include suitable anti-foaming agents known by anyone skilled in the art.

The accumulation of the substantially pure carotenoid inside the bacterial cells may be influenced by several factors, including but not limited to stress factors such as the addition of a slowly metabolisable carbon source, the addition of precursors of the carotenoid biosynthetic pathways, the addition of growth inhibiting compounds, changes of culture pH, changes of temperature, changes of salt concentration, changes of carbon source concentration, changes of concentration of nitrogen source concentration, changes of the carbon/nitrogen ratio, changes of dissolved oxygen concentration.

Said stress factors can be used individually or in any combination. Said stress factors can be used once or repeatedly during the fermentation time course.

The proportions of the nutrients can also be determined as function of the growth needs of the micro-organism and the production levels. The addition of medium components can be controlled in such a way that they are present in suitable ranges, between minimum and maximum concentration levels. For example, excessive glucose concentrations lead to growth inhibition, while limiting glucose concentrations lead to decreased productivities.

Similarly, excessive salt concentrations may result in an increase of the ionic strength of the medium which is deleterious to the culture, while limiting salt concentrations may deprive the culture from essential co-factors. Additionally, the dissolved oxygen concentration may affect the balance between hydroxylated and non-hydroxylated carotenoids, thus affecting the purity of the produced carotenoids.

The present invention for the production of carotenoids, particularly substantially pure carotenoids, most preferably substantially pure beta-carotene, with any naturally occurring bacterial strain, further comprises suitable purification steps for the separation of biomass and subsequent extraction and purification of the carotenoids, particularly substantially pure carotenoids, most preferably substantially pure beta-carotene, from the biomass produced during the fermentation step.

Preferably, the purification steps according to the present invention do not involve cell disruption and comprise the direct extraction of the carotenoids, particularly of the substantially pure carotenoids, most preferably substantially of the pure beta-carotene, from the biomass produced during the fermentation step with a suitable natural solvent or a mixture of suitable natural solvents, eventually preceded by a washing step, followed by extraction to another natural solvent or mixture of natural solvents and finally treating the thus obtained extract by means of final polishing steps.

The separation of the biomass from the whole fermentation broth can be carried out using established operations of filtration, using the current filter technologies, either strips, rotary, presses, organic or inorganic membranes in modules, in which the barrier constituted by the filtering material retains the biomass and allows the liquid to pass without the biomass; or centrifugation, in which, making use of the different densities between the broth and the biomass in an equipment such as a centrifuge, decanter or similar is used, in which the heavier phase is concentrated and separated from the liquid phase with the lowest possible quantity of biomass; in such a way that the losses of biomass are minimized.

These steps can additionally be coupled to a washing step in which an appropriate washing solution, such as but not limited to water, saline, or natural organic solvent is added to and then separated form the retained biomass. Although the substantially pure carotenoid is intracellular, the process of the present invention has the important advantage over processes for the production of carotenoids using micro-organisms of allowing for the direct extraction of the substantially pure carotenoid from the biomass without any cell disruption steps.

In this way, the process of the present invention does not need to separate the carotenoids from the fraction of bulk cellular components generated when the cell is disrupted to release the intracellular compounds of interest.

For the extraction of the substantially pure carotenoid from the biomass prepared as described here, different organic solvents can be used. This invention relates to the use of food-grade solvents considered as natural or mixtures thereof which present reasonably high solubility for the carotenoid components, which are admissible for both pharmaceutical and food applications. These solvents can be recovered and reused. Following this extraction step, biomass separation from the extract is carried out in order to remove spent biomass and biomass debris from the extract.

As before, this step can be performed in any liquid/solid separation unit operation such as filtration or centrifugation or decantation. The clarified extract is then further processed through a liquid-liquid extraction unit operation wherein a hydrophobic solvent or a mixture of hydrophobic solvents is used to separate the substantially pure carotenoid from any membrane lipids that might have been co-extracted from the biomass.

As membrane lipids are bi-polar, while the substantially pure carotenoid, particularly the substantially pure non-hydroxylated carotenoid, is apolar, the former will preferably partition to the ketone/alcohol phase, while the later will preferably partition to the hydrophobic phase. Water can be added to the mixture to further improve partitioning of unwanted compounds.

The thus purified substantially pure carotenoid is then crystallized using techniques known by anyone skilled in the art, such as adding to the extract compounds in which the substantially pure carotenoid is substantially insoluble, then allowing the crystals to form, followed by crystal recovery by filtration or centrifugation and finally crystal drying under vacuum for removal of the residual solvents.

### DETAILED DESCRIPTION OF THE INVENTION

The present invention describes naturally occurring bacterial strains constitutively over-producing carotenoids, particularly beta-carotene, or mutants thereof, and the process of production of carotenoids, preferably beta-carotene, in improved conditions of fermentation using cheap and renewable raw materials, purifying and isolating a specific crystalline carotenoid of high purity from the fermentation broth previously obtained for its use in the feed, food, cosmetic and pharmaceutical sectors.

### 1. Bacterial Strains

In this invention isolates of bacteria belonging to the following genera are preferably used:

Mycobacterium, Pseudomonas, Dietzia, Flavobacterium, Paracoccus, Rhodococcus, Blastomonas, Sphingomonas, Brevibacterium, Erwinia, Pantoea, Agrobacterium, Paracoccus, Erythrobacter, Xanthobacter, Sphingobacteria, Rhodobacter, Gordonia, Rubrobacter, Arthrobacter, Novosphingobium, Nocardia, Corynebacterium, Streptomyces, Enterobacteriaceae, Thermobifida, Enterobacter, Brevundimonas, Roseiflexus, Sphingopyxis, Aurantimonas, Photobacterium, Robiginitalea, Polaribacter, Tenacibaculum, Parvularcula, Deinococcus, Chloroflexus genera, more preferably bacteria belonging to the Mycobacterium, Pseudomonas, Dietzia, Flavobacterium, Paracoccus, Rhodococcus, Blastomonas, Sphingomonas, Brevibacterium, Erwinia, Pantoea, Agrobacterium, Paracoccus, Erythrobacter, Xanthobacter, Sphingobacteria, Rhodobacter, Gordonia, Rubrobacter, Arthrobacter, Novosphingobium, Nocardia, Corynebacterium, Streptomyces genera, most preferably bacteria belonging to the Mycobacterium, Pseudomonas, Dietzia, Flavobacterium, Paracoccus, Rhodococcus, Blastomonas, Sphingomonas, Brevibacterium, Erwinia, Pantoea, Paracoccus, Erythrobacter, Xanthobacter, Rhodobacter, Gordonia, Novosphingobium, Nocardia, Corynebacterium genera, of utmost preference are bacteria belonging to the Mycobacterium, Pseudomonas, Dietzia, Flavobacterium, Paracoccus, Rhodococcus, Blastomonas and Sphingomonas genera, particularly bacteria belonging to the Sphingomonas genus. Among these bacteria, a particular Sphingomonas sp. strain isolated form soil, characterized by having the 16S rRNA gene sequence SEQ ID 1 is preferred.

Bacteria derived from the natural isolates using the mutagenic and selection method of the invention are also preferably used. The strain Sphingomonas M63Y obtained using the selection and mutagenic method of this invention that over-produces beta-carotene with high specificity is particularly preferred. This improved strain obtained using the selection and mutagenic method of this invention presents the following characteristics:

### Morphological Characteristics

Strain Sphingomonas M63Y is Gram-negative, rod shaped and non-spore forming.

### Physiological Characteristics

On nutrient agar, the strain forms round, smooth, orange colonies. Strain M63Y is able to grow between 20 and 30° C., with optimum growth at 27° C. Other physiological characteristics as determined by the API 20NE kit (Biomérieux, France) are:

TABLE 1

| | |
|---|---|
| Nitrate reduction | negative |
| Tryptophane conversion | negative |
| Glucose acidification | negative |
| Arginine dihydrolase | negative |
| Urease | negative |
| Esculin hydrolysis | positive |
| Gelatine hydrolysis | negative |
| Beta-galactosidase | positive |
| Cytochrome oxidase | positive |

Assimilation of:

TABLE 2

| | |
|---|---|
| Glucose | positive |
| Arabinose | positive |
| Mannose | positive |
| Mannitol | positive |
| N-acetyl-glucosamine | positive |
| Maltose | positive |
| Gluconate | positive |
| Caprate | positive |
| Adipate | positive |
| Malate | positive |
| Citrate | positive |
| Phenyl-acetate | positive |

### Chemotaxonomic Characteristics

Strain M63Y contains meso-diaminopimelic acid (meso-Dpm), typical of the peptidoglycan type A1γ. The major isoprenoid quinone is ubiquinone-10, which constitutes 80% of the total quinones.

Cellular fatty acid composition:

TABLE 3

| Fatty acid | Ratio (%) |
|---|---|
| 14:0 | 2.0 |
| 14:0 2OH | 6.3 |
| 16:1 w7c/15 iso 2OH | 10.5 |
| 16:1 w5c | 1.4 |
| 16:0 | 12.1 |
| 15:0 2OH | 0.2 |
| 17:1 w8c | 0.3 |
| 17:1 w6c | 4.4 |
| 17:0 | 0.4 |
| 16:1 2OH | 0.3 |
| 18:1 w7c | 53.1 |
| 18:1 w5c | 1.4 |
| 18:0 | 0.9 |
| 11 methyl 18:1 w7c | 6.4 |
| 19:0 cyclo w8c | 0.3 |

The strain produces polar lipids, including sphingoglycolipids. The major carotenoid is beta-carotene, but other carotenoids are also detected. The G+C content of the DNA of the strain M63Y is 66.6 mol %.

### Phylogenetic Analysis

Approximately 95% of the 16S rRNA gene sequence of strain M63Y were determined by direct sequencing of PCR-amplified 16S rDNA (SEQ 2). Genomic DNA extraction, PCR mediated amplification of the 16S rDNA and purification of the PCR product was carried and the purified PCR products were sequenced. Sequence reactions were submitted to electrophoresis and the resulting sequence data was aligned and compared with representative 16S rRNA gene sequences of organisms belonging to the Alphaproteobacteria. The 16S rRNA gene similarity values were calculated by pairwise comparison of the sequences within the alignment. Strain M63Y was closely related to species belonging to the genus Sphingomonas and formed a cluster with those species. The sequence of strain M63Y showed highest levels of similarity with Sphingomonas oligophenolica (98.5%) and laid in the same cluster as Sphingomonas echinoides (97.9% similarity). It exhibited a 98% similarity with the original Sphingomonas isolate. DNA-DNA hybridization of strain M63Y against Sphingomonas oligophenolica and Sphingomonas echinoides was performed and the percentage DNA-DNA similarity were, respectively, 16% and 3%.

These results suggest that the improved strain obtained using the method of this invention is a new Sphingomonas strain.

### 2. Method for the Production and Selection of Mutants of Naturally Occurring Bacterial Strains

The present invention provides a method for the improvement of naturally occurring bacterial strains based on the production of mutants using classical mutagenic techniques or by spontaneous mutation coupled to a series of phenotypical tests aiming at identifying strains over-producing carotenoids, preferably over-producing a substantially pure carotenoid, most preferably over-producing substantially pure beta-carotene.

### 2.1. Strain Improvement

Naturally occurring carotenoid accumulating bacterial strains isolated from natural sources, such as but not limited to soil, or mutants thereof are cultivated as described elsewhere (Silva et al., 2004).

Cells from an actively growing culture of selected strains are collected by centrifugation (15000 g, 30 s) and treated with 15 mM phosphate buffer, pH 6.5, containing ethyl methanesulfonate (EMS) or nitrosoguanidine (NTG) at a concentration between 0 and 40 μg/mL, preferably between 0 and 20 μg/mL, most preferably between 0 and 15 μg/mL, for a time period less or equal than 2 h, preferably less or equal than 1 hour, most preferably less or equal than 30 minutes, at a temperature suitable for the growth of the strain, to achieve mortality rates higher than 90%, preferably close to 99%.

The thus treated cells are washed twice with saline, and allowed to recover in any standard liquid culture medium for at least 3 h, preferably at least overnight.

Dilutions of the thus obtained cultures are spread out on standard solid culture medium and the plates incubated at a temperature suitable for the growth of the strains during a period of time suitable for colony formation. Samples of untreated cultured cells are also plated to serve as reference. The dilutions of the cultures are such that less than 250 colonies per plate are obtained, preferably less than 100 colonies per plate are obtained, most preferably close to 50 isolated colonies per plate are obtained.

### 2.2. Selection of Improved Strains

Selection was first performed by visual phenotypic analysis, on the basis of colony colour and morphology. Colonies that did not show any colour change as compared to colonies of the parent strains plated from reference samples, or that were slimy were rejected.

Colonies with altered colour are grown in liquid medium until fermentation completion (about 72 hours) for analysis of biomass, total carotenoid and beta-carotene production. The turbidity of the fermentation broth is measured at 600 nm and correlated to biomass concentration.

For carotenoid analysis, cells are collected by centrifugation (15000 g, 30 s), and the pellet is resuspended in saline, followed by centrifugation (15000 g, 30 s). The washed cell pellet is extracted with a suitable solvent or solvent mixture at ambient temperature. Suitable solvents include but are not limited to, methanol, acetone and dichloromethane or combinations thereof.

The extracts are centrifuged (15000 g, 30 s) and carotenoids are analyzed by thin layer chromatography (TLC) and HPLC as described elsewhere (Silva et al., 2004).

Criteria for selecting mutant strains with improved characteristics are an increase of at least 5% of accumulated total carotenoids or single carotenoid, preferably beta-carotene per unit of biomass or unit of culture liquid or an increase of at least 5% of the accumulated fraction of single carotenoid, preferably beta-carotene, in relation to total carotenoids. The procedure herein described for obtaining mutants with improved single carotenoid, preferably beta-carotene, production can be also applied to obtained mutants so as to improve their performance even further.

3. Process for the Production of Carotenoids with Development of Improved and Controlled Conditions of Fermentation and Purification Using a Naturally Occurring Bacterial Strain or a Mutant Thereof

Another object of the present invention is a process for the production of carotenoids, particularly substantially pure carotenoids, most preferably substantially pure beta-carotene, with any naturally occurring bacterial strain or mutant strains thereof as defined hereinbefore, consisting of culturing said bacterial strain in a liquid fermentation medium, applying defined strategies for the control inter alia of pH, dissolved oxygen and carbon source concentration during the fermentation; separating the biomass; extracting and purifying the carotenoids, particularly substantially pure carotenoids, most preferably substantially pure beta-carotene, from the biomass produced during the fermentation step.

The fermentation step can be performed in any culture medium containing one or more sources of carbon, one or more sources of nitrogen and mineral salts. The carbon sources that can be used as single or complex nutrients include carbohydrates (such as but not limited to glucose, sucrose, fructose, lactose, starches, either purified or in bulk mixtures containing said carbohydrates, such as but not limited to corn steep liquor and cheese whey), edible oils, preferably vegetable oils, such as, but not limited to for example olive oil, soybean oil, rapeseed oil, palm oil, peanut oil, canola oil, or any other assimilable carbon and/or energy source, such as, but not limited to for example glycerol and lipids. Glucose is preferably used as main carbon source, at a concentration preferably kept between 40 g/L and 0 g/L, preferably between 20 g/L and 0 g/L, most preferably between 10 g/L and 0 g/L.

Nitrogen sources used in the fermentation step include organic and inorganic sources, such as but not limited to for example soybean hulls, soybean flour, corn flour, yeast extract, cotton flour, peptones, casein, amino acids, ammonium sulphate, ammonium chloride, ammonium nitrate, ammonium hydroxide. Adequate carbon/nitrogen ratios can be controlled throughout the fermentation to values adequate to each fermentation stage. This ratio is preferably set to be in the range 10-20 carbon equivalents to nitrogen equivalents, most preferably in the range 12-15 carbon equivalents to nitrogen equivalents.

Mineral salts used in the fermentation step include but are not limited to phosphates, sulphates, chlorides or molybdates of cations such as but not limited to sodium, potassium, ammonium, calcium, copper, iron, manganese, magnesium or zinc. Phosphate concentration is preferably kept between 1 g/L and 10 g/L, most preferably between 2 g/L and 4 g/L; magnesium concentration is preferably kept between 0.01 g/L and 0.2 g/L, most preferably between 0.05 g/L and 0.15 g/L.

The fermentation step is carried out in aerobic conditions and submerged culture. The temperature ranges from 20° C. to 37° C., preferably between 22° C. and 31° C., most preferably between 24° C. and 28° C.

During the fermentation step, the dissolved oxygen of the culture is controlled at levels between 100% and 0% air saturation, preferably at levels between 50% and 0.5% air saturation, most preferably at levels between 30% and 1% air saturation. The dissolved oxygen concentration is controlled by means of suitably combining the effects of regulating the air flow rate into the culture broth and the stirring speed of the turbines or impellers used. As an alternative the air flow can be enriched with oxygen. The control set-point may be a constant value or may be a value varying over time. When the accumulation of beta-carotene is desired, the oxygen concentration should be kept below 50% air saturation, preferably below 30% air saturation, most preferably below 10% air saturation, even more preferably below 5% air saturation.

The pH during the fermentation step is controlled by means of the addition of acid and/or alkali and/or carbon source within the range of 6.0-8.0, preferably 6.4-7.6. The start of the control depends on the growth pattern of the culture, but it generally takes place after between 1 and 48 hours of fermentation, preferably between 10 and 28 hours, or upon the first occurrence of drop or rise of the pH of the culture, as consequence of carbon source uptake or depletion, respectively. The control set-point may be a constant value or may be a value varying over time.

The process of the present invention for the production of carotenoids, particularly substantially pure carotenoids, most preferably substantially pure beta-carotene, with any naturally occurring bacterial strain, further comprises any purification steps for the separation of biomass and subsequent extraction and purification of the carotenoids, particularly substantially pure carotenoids, most preferably substantially pure beta-carotene, from the biomass produced during the fermentation step.

Purification unit operations and their sequences can be readily chosen by anyone skilled in the art. Preferably, the purification steps of the process according to the present invention comprises the direct extraction of the carotenoids, particularly of the substantially pure carotenoids, most preferably substantially of the pure beta-carotene, from the biomass produced during the fermentation step with a suitable natural solvent or a mixture of suitable natural solvents, eventually preceded by washing, followed by extraction to another natural solvent or mixture of natural solvents and finally treating the thus obtained extract by means of final polishing steps.

The separation of the biomass from the whole fermentation broth can be carried out using established operations of filtration, using the current filter technologies, either strips, rotary, presses, organic or inorganic membranes in modules, in which the barrier constituted by the filtering material retains the biomass and allows the liquid to pass without the biomass; or centrifugation, in which, making use of the different densities between the broth and the biomass in an equipment such as a centrifuge, decanter or similar is used, in which the heavier phase is concentrated and separated from the liquid phase with the lowest possible quantity of biomass; in such a way that the losses of biomass are minimized. These steps can additionally be coupled to a washing step in which an appropriate washing solution, such as but not limited to water, saline, or natural organic solvent is added to and then separated form the retained biomass. The resulting biomass contains more than 80% of the carotenoids produced in the fermentation, preferably more than 95% and most preferably more than 99% of the carotenoids produced in the fermentation.

Although the substantially pure carotenoid is intracellular, the purification steps of the process of the present invention comprise the direct extraction of the substantially pure carotenoid from the biomass of a bacterial strain selected by the method of this invention without any prior cell disruption being needed. For this direct extraction of the substantially pure carotenoid from the biomass, different organic solvents can be used. This invention relates to the use of food-grade solvents considered as natural or mixtures thereof which present reasonably high solubility for the carotenoid components, which are admissible for both pharmaceutical and food applications. Preferably, a mixture of a ketone and an alcohol is used, most preferably a mixture of acetone and ethanol, most preferably a mixture of acetone and methanol is used, at a ketone/alcohol ratio of 0/1 to 1/0, preferably at a ketone/alcohol ratio of 1/9 to 9/1, most preferably at a ketone/alcohol ratio of 2/7 to 7/2.

The extraction temperature varies between room temperature and that of the boiling point of the solvents, preferably between room temperature and 80° C., most preferably at room temperature.

The extraction time will be the minimum necessary to achieve solubilisation of the substantially pure carotenoid, between 1 second and 1 hour, preferably between 1 minute and 15 minutes. The quantity of solvent or of mixture of solvents used depends on the temperature and the ratio between mass of the substantially pure carotenoid and the mass of biomass, ranging between 5 ml/g and 100 ml/g.

The number of extractions varies from 1 to 3, preferably less than 3. Continuous extraction can be used with appropriate residence times.

The yield of the extraction of the substantially pure carotenoid is greater than 85%, preferably greater than 90% and more preferably greater than 95%.

Following this extraction step, biomass separation from the extract is carried out in order to remove spent biomass and biomass debris from the extract. As before, this step can be performed in any liquid/solid separation unit operation such as filtration or centrifugation or decantation.

The clarified extract is then further processed through a liquid-liquid extraction unit operation wherein a hydrophobic solvent or a mixture of hydrophobic solvents is used to separate the substantially pure carotenoid from any membrane lipids that might have been co-extracted from the biomass. As membrane lipids are bi-polar, while the substantially pure carotenoid, particularly the substantially pure non-hydroxylated carotenoid, is apolar, the former will preferably partition to the ketone/alcohol phase, while the later will preferably partition to the hydrophobic phase. Water can be added to the mixture to further improve partitioning of unwanted compounds. Preferably, solvents such as but not limited to hexane and tert-butylmethyl ether are used.

The thus purified substantially pure carotenoid is then crystallized using processes known by anyone skilled in the art, such as adding to the extract compounds in which the substantially pure carotenoid is substantially insoluble, then allowing the crystals to form, followed by crystal recovery by filtration or centrifugation and finally crystal drying under vacuum for removal of the residual solvents.

The following examples describe the present invention in detail and without limitation.

### Example 1

### Derivation of Beta-Carotene Over-Producing Mutants by Chemical Mutagenesis of a Naturally Occurring Sphingomonas Strain Isolated from Soil

Soil samples were collected from various sites in the Greater Lisbon area, Portugal. The samples were suspended in water and serial dilutions were spread on agar plates. Yellow and orange-coloured colonies were isolated and replated 4 times to confirm phenotypic stability and the absence of contaminant strains. A period of incubation in the dark was used to confirm that the colour production was constitutive and not photoinduced.

The strain was identified as Sphingomonas sp. using API 20NE kits (24-48 hour identification of gram-negative non-Enterobacteriaceae kits, form Biomérieux, France) and by 16S rRNA gene sequencing (SEQ ID: 1).

The isolated strain had a specific growth rate of 0.18 h⁻¹, it constitutively accumulated carotenoids at a concentration of 1.7 mg/g dry cell weight, of which 29% was beta-carotene.

The strain was grown in the medium used by Silva et al. (2004). The cells from the growing culture were collected, during the exponential growth phase, by centrifugation (15000 g, 30 s) and treated with 15 mM phosphate buffer, pH 6.5, containing EMS at a concentration of 40 μL/mL, for a time period of 30 minutes, at room temperature. The thus treated cells were washed twice with saline, and allowed to recover in any standard liquid culture medium for 3 h.

A 1:10⁷-1:10⁸ dilution of the cultures was spread out on 50 plates containing standard solid culture medium. The plates were incubated at 28° C. during three days.

The cells that presented a visually detectable change in colour intensity were analysed for total carotenoid content and beta-carotene purity. A strain was obtained, referred as strain EMS1 that accumulated 3.8 mg/g dry cell weight, of which 24% was beta-carotene. This strain was submitted to another mutagenesis cycle such as that described above using EMS as mutagenic agent.

The cells that presented a visually detectable change in colour intensity were analysed for total carotenoid content and beta-carotene purity. A strain was obtained, referred as strain EMS2 that accumulated 3.3 mg/g dry cell weight, of which 71% was beta-carotene.

Thus, although the total carotenoids produced per biomass unit were slightly different, this mutant accumulated far more beta-carotene. This mutant was selected to perform a further mutagenic cycle, under de same conditions as above.

A total of 69 colonies with altered phenotype in terms of colour development as compared to the parent strain were obtained. Cells from each obtained colony were incubated in liquid culture medium and grown in the same conditions as described above. After 3 days, the cultures were analysed for optical density, total carotenoids and beta-carotene concentration.

From these measurements, the beta-carotene purity was calculated as the concentration of beta-carotene divided by the concentration of total carotenoids, and the cellular content of beta-carotene was obtained by dividing the concentration of beta-carotene by the biomass concentration.

All cultures were scored according to the concentration of biomass and beta-carotene, the purity of beta-carotene and the cellular content of beta-carotene (Table 1). A score of 69 was attributed to the best performing strain in each parameter, a score of 68 is attributed to the second best performing strain in each parameter, and so on so that the worst performing strain in each parameter is given a score of 0. The score shown in Table 2 given to each mutant is obtained by adding the scores given to that mutant in each parameter.

Table 1. Performance of the mutants obtained after 3 cycles of mutagenesis on an isolated Sphingomonas strain constitutively producing carotenoids, using the mutagenic method herewith described, after 3 days of culture in shaken flasks. The top performing mutants in each parameter are underlined. [OD600 mm: biomass concentration expressed in optical density units measured at 600 nm; % B: purity of beta-carotene with respect to total carotenoids; B (mg/L): beta carotene concentration; B (mg/g): cellular content of beta-carotene].

TABLE 4

| Mutant | OD600 nm | % B | B (mg/L) | B (mg/g) | Score |
|---|---|---|---|---|---|
| EMS2 | 12.8 | 61.7 | 9.3 | 2.4 | 111 |
| M2 | 13.6 | 58.8 | 7.9 | 1.9 | 41 |
| M3 | 13.8 | 57.2 | 7.9 | 1.9 | 38 |
| M4 | 14.8 | 60.5 | 8.8 | 2.0 | 104 |
| M5 | 13.3 | 63.7 | 8.6 | 2.2 | 101 |
| M6 | 14.8 | 62.8 | 8.5 | 1.9 | 127 |
| M7 | 14.8 | 54.8 | 8.6 | 1.9 | 77 |
| M8 | 13.4 | 63.4 | 9.2 | 2.3 | 125 |
| M9 | 6.1 | 4.3 | 0.2 | 0.1 | 1 |
| M10 | 14.7 | 61.8 | 9.5 | 2.2 | 135 |
| M11 | 14.6 | 59.4 | 10.0 | 2.3 | 133 |
| M12 | 14.7 | 60.6 | 9.7 | 2.2 | 134 |
| M13 | 12.6 | 60.0 | 8.3 | 2.2 | 50 |
| M14 | 15.0 | 61.1 | 9.9 | 2.2 | 148 |
| M15 | 14.3 | 60.0 | 9.6 | 2.2 | 118 |
| M16 | 13.2 | 60.3 | 8.9 | 2.2 | 74 |
| M17 | 13.9 | 61.8 | 9.4 | 2.2 | 112 |
| M18 | 12.3 | 62.1 | 10.6 | 2.9 | 174 |
| M19 | 14.1 | 60.1 | 10.8 | 2.6 | 163 |
| M20 | 14.1 | 65.1 | 10.6 | 2.5 | 205 |
| M21 | 14.0 | 63.1 | 10.8 | 2.6 | 191 |
| M22 | 14.2 | 60.4 | 10.3 | 2.4 | 152 |
| M23 | 15.2 | 57.3 | 10.2 | 2.2 | 133 |
| M24 | 13.9 | 59.0 | 9.4 | 2.2 | 84 |
| M25 | 14.5 | 61.8 | 10.6 | 2.4 | 185 |
| M26 | 14.9 | 58.4 | 9.2 | 2.1 | 98 |
| M27 | 14.3 | 60.4 | 9.8 | 2.3 | 136 |
| M28 | 13.6 | 62.1 | 9.4 | 2.3 | 126 |
| M29 | 2.5 | 58.1 | 3.5 | 4.6 | 75 |
| M30 | 13.7 | 62.0 | 9.7 | 2.4 | 137 |
| M31 | 11.4 | 63.7 | 9.5 | 2.8 | 160 |
| M32 | 11.4 | 63.0 | 8.5 | 2.5 | 110 |
| M33 | 10.9 | 62.1 | 8.9 | 2.7 | 124 |
| M34 | 12.0 | 62.5 | 9.9 | 2.8 | 161 |
| M35 | 11.1 | 60.5 | 7.9 | 2.4 | 69 |
| M36 | 14.0 | 59.9 | 9.9 | 2.4 | 119 |
| M37 | 12.8 | 59.1 | 10.3 | 2.7 | 126 |
| M38 | 11.8 | 58.5 | 9.4 | 2.6 | 94 |
| M39 | 15.0 | 61.5 | 11.2 | 2.5 | 199 |
| M40 | 11.9 | 59.5 | 9.2 | 2.6 | 92 |
| M41 | 14.0 | 60.0 | 9.5 | 2.2 | 100 |
| M42 | 14.1 | 62.1 | 11.2 | 2.6 | 199 |
| M43 | 14.1 | 59.9 | 9.7 | 2.3 | 117 |
| M44 | 14.1 | 61.6 | 10.2 | 2.4 | 151 |
| M45 | 14.9 | 62.0 | 10.5 | 2.4 | 186 |
| M46 | 14.1 | 62.3 | 7.4 | 1.8 | 91 |
| M47 | 13.9 | 61.2 | 9.6 | 2.3 | 120 |
| M48 | 12.3 | 61.7 | 8.3 | 2.2 | 78 |
| M49 | 13.5 | 61.1 | 9.0 | 2.2 | 83 |
| M50 | 13.8 | 62.6 | 9.6 | 2.3 | 137 |
| M51 | 14.3 | 63.1 | 10.7 | 2.5 | 202 |
| M52 | 14.6 | 59.7 | 12.3 | 2.8 | 199 |
| M53 | 14.3 | 61.8 | 11.0 | 2.6 | 192 |
| M54 | 14.1 | 63.7 | 11.7 | 2.8 | 221 |
| M55 | 14.1 | 64.8 | 11.8 | 2.8 | 228 |
| M56 | 14.8 | 63.0 | 11.5 | 2.6 | 222 |
| M57 | 14.5 | 61.5 | 12.0 | 2.7 | 209 |
| M58 | 15.0 | 61.7 | 12.0 | 2.7 | 221 |
| M59 | 13.8 | 63.8 | 11.8 | 2.8 | 218 |
| M60 | 14.1 | 60.4 | 10.5 | 2.5 | 150 |
| M61 | 14.4 | 61.6 | 10.5 | 2.4 | 169 |
| M62 | 9.2 | 65.5 | 9.7 | 3.5 | 172 |
| M63 | 15.8 | 63.7 | 12.7 | 2.7 | 256 |
| M64 | 14.2 | 66.3 | 12.7 | 3.0 | 250 |
| M65 | 13.4 | 63.6 | 10.6 | 2.6 | 179 |
| M66 | 14.1 | 64.6 | 11.5 | 2.7 | 218 |
| M67 | 12.1 | 67.0 | 5.8 | 1.6 | 83 |
| M68 | 11.3 | 61.0 | 9.1 | 2.7 | 105 |
| M69 | 13.1 | 55.9 | 8.8 | 2.2 | 52 |

The data obtained for the strain EMS2 are different from those reported above, simply because these were obtained form a 3-day old culture in liquid medium, instead of colonies taken form an agar plate.

Mutant M63 yielded the best overall result and scored highest both on the biomass concentration attained after 3 days of culture and on the concentration of beta-carotene. It provided major improvements over the isolated strain, such as a 23% increase on the biomass concentration, a 37% increase on total beta-carotene produced, a 12.5% increase on the intracellular concentration of beta-carotene with slightly higher beta-carotene purity.

### Example 2

### Selection of Spontaneous Mutants Over-Producers of Beta-Carotene

M63 cells (obtained in Example 1) were repeatedly replated until a phenotypical change was observed, such as the colour of the formed colonies.

M63, which produces deep orange colonies, originated yellow colonies after successive replating, designated M63Y. M63 and M63Y cells were incubated in liquid culture medium as in Example 1 and grown during 5 days in the same conditions used above. The cultures were periodically sampled and analysed for optical density, total carotenoids and beta-carotene concentration. From these measurements, the beta-carotene purity was calculated as the concentration of beta-carotene divided by the concentration of total carotenoids, and the cellular content of beta-carotene was obtained by dividing the concentration of beta-carotene by the biomass concentration. The maximum value for each of these parameters obtained during the time course of the cultures is presented in Table 3.

Table 3. Performance of the mutants M63Y obtained from M63 through phenotypical selection upon successive replating [OD600 nm: biomass concentration expressed in optical density units measured at 600 nm; % B: purity of beta-carotene with respect to total carotenoids; B (mg/L): beta carotene concentration; B (mg/g): cellular content of beta-carotene].

TABLE 5

| Mutant | OD600 nm | % B | B (mg/L) | B (mg/g) |
|---|---|---|---|---|
| M63 | 21.2 | 64.8 | 14.4 | 3.09 |
| M63Y | 19.2 | 78.1 | 24.1 | 4.82 |

The mutant strain M63Y, obtained from strain M63 using the mutation and selection method of the present invention, hereinbefore described, shows an enhanced beta-carotene purity (78% with respect to total carotenoids) when compared to the parent strain M63, while yielding a higher intracellular content of beta-carotene. The colour change of the cells from orange to yellow can be explained through the increase of the relative amount of beta-carotene with respect to red carotenoids, such as lycopene. 16S rRNA gene sequence analysis of strain M63Y was performed (SEQ ID: 2). The sequence was compared to data obtained from the European Molecular Biology Laboratory database or the Ribosomal Database Project and it was concluded that the M63Y strain represents a new species within the genus Sphingomonas.

### Example 3

### Effect of Dissolved Oxygen on the Production of Beta-Carotene

M63Y cells (obtained in Example 2) were grown overnight in shake flasks containing 75 mL of the liquid culture medium used in Example 1, using an orbital shaker (200 rpm, 27° C.). These cultures were used as inoculum to bioreactors containing 2 L of culture medium (glucose, 10 g/L; yeast extract 10 g/L; 10 g/L glycerol).

All cultures were carried out at constant pH (6.75) and at different constant levels of dissolved oxygen concentration (% DO: 20%, 10%, 5% and 2% of oxygen saturation concentration in equilibrium with atmospheric air).

TABLE 4. Production of beta-carotene in bioreactors by culturing strain M63Y at different levels of dissolved oxygen concentration. [OD600 nm: biomass concentration expressed in optical density units measured at 600 nm; % B: purity of beta-carotene with respect to total carotenoids; B (mg/L): beta carotene concentration; B (mg/g): cellular content of beta-carotene; TC (mg/L): concentration of total carotenoids; % DO, dissolved oxygen concentration as percentage of oxygen saturation].

TABLE 6

| % DO | OD600 nm | TC (mg/L) | B (mg/L) | B (mg/g) | % B |
|---|---|---|---|---|---|
| 20% | 21.5 | 24.7 | 9.9 | 1.6 | 50.0 |
| 10% | 20.1 | 21.7 | 11.4 | 2.1 | 52.7 |
| 5% | 21.2 | 23.9 | 14.4 | 3.1 | 64.8 |
| 2% | 22.0 | 23.3 | 17.7 | 3.39 | 75.6 |

Table 4 shows that beta-carotene accumulation is favoured at low dissolved oxygen concentrations. This is explained by the fact that beta-carotene is a non-hydroxylated carotenoid. When oxygen is present, beta-carotene can be converted by the action of hydroxylases to a hydroxylated compound, downstream in the carotenogenic pathway.

### Example 4

### Stress-Induced Beta-Carotene Production

### Example 4a

### Batch Cultures

M63Y cells (obtained in Example 2) were grown overnight in shake flasks containing 75 mL of the liquid culture medium used in Example 1, using an orbital shaker (200 rpm, 27° C.). These cultures were used as inoculum to bioreactors containing 2 L of culture medium (glucose, 10 g/L; yeast extract 10 g/L; 10 g/L glycerol). All cultures were carried out at 2% dissolved oxygen concentration. During the time course of two of the three fermentations, the pH was increased to 7.40 at different time points.

TABLE 5. Effect of pH increase during the time course of the fermentation of strain M63Y in the production of beta-carotene in bioreactors. [OD600 nm: biomass concentration expressed in optical density units measured at 600 nm; % B: purity of beta-carotene with respect to total carotenoids; B (mg/L): beta carotene concentration; B (mg/g): cellular content of beta-carotene; TC (mg/L): concentration of total carotenoids].

TABLE 7

| Time of pH | | | | | |
|---|---|---|---|---|---|
| increase | OD600 nm | TC (mg/L) | B (mg/L) | B (mg/g) | % B |
| | 22.0 | 23.3 | 17.7 | 3.39 | 75.6 |
| 21.25 h | 19.2 | 27.4 | 22.1 | 6.05 | 79.2 |
| 13.75 h | 13.5 | 18.2 | 14.6 | 5.54 | 81.1 |

Table 5 shows that a pH increase during the time-course of the fermentation increases the intracellular concentration of beta-carotene. In both fermentations in which the pH was increased the final optical density was lower than that obtained when no pH increase was performed. The lowest optical density was obtained when the pH increase was performed sooner, showing that this increase imposed a stress on the cells. Beta-carotene and carotenoids in general are recognised as being involved in stress-response cellular mechanisms. Table 5 shows that the intracellular concentration of beta-carotene is higher in the cells submitted to pH-induced stress.

### Example 4b

### Fed-Batch Culture

A three-stage fermentation was carried in which the first 14.5 h were performed batchwise in 2 L growth medium at a pH of 6.50, after that, the pH was increased to 7.30 and after 27 h of fermentation 200 mL of growth medium containing 40 g/L glucose was fed to the culture at a constant feeding rate so as to maintain glucose at limiting concentrations. This resulted in the production of 15.2 optical density units of biomass, 47.7 mg/L total carotenoids, with a 91.6% purity of beta-carotene with respect to total carotenoids and an intracellular concentration of beta-carotene of 11.4 mg/g.

### REFERENCES

V. Böhm, N. L. Puspitasari-Nienaber, M. G. Ferruzzi, S. J. Schwartz (2002) Trolox equivalent antioxidant capacity of different geometrical isomers of a-carotene, b-carotene, lycopene, and zeaxanthin. Journal of Agricultural and Food Chemistry, 50: 221-226.
M. A. Borowitzka (1990) The mass culture of Dunaliella salina. In: Technical resource papers—Regional workshop on the culture and utilization of seaweeds—volume II, Food and Agriculture Organization.
P. Buzzini (2004) An optimization study of carotenoid production by Rhodotorula glutinis DBVPG 3853 from substrates containing concentrated rectified grape must as the sole carbohydrate source. Journal of Industrial Microbiology and Biotechnology, 24: 41-45.
A. Ciegler (1965) Microbial carotenogenesis. Adv. Appl. Microbiol. 7: 1-34.
H. L. David (1974a) Biogenesis of beta-carotene in Mycobacterium kansasii, Journal of Bacteriology, 119: 527-533.
H. L. David (1974b) Carotenoid pigments of Mycobacterium kansasii. Appl Microbiol, 28:696-699.
M. A. Ingraham, C. A. Baumann (1933) The relation of microorganisms to carotenoids and vitamin A-I. The occurrence of carotene in bacteria, Journal of Bacteriology, 28: 31-40.
E. A. Iturriaga, T. Papp, J., Breum, J., Arnau, A. P., Eslava (2005) Strain and Culture Conditions Improvement for β-Carotene Production with Mucor. Methods in Biotechnology, Vol. 18: Microbial Processes and Products, pp. 199-216. Ed. J. L. Barredo, Humana Press Inc., Totowa, N.J.
K. S. Kang, G. T. Veeder, P. J. Mirrasoul, T. Kaneko, I. W. Cottrell (1982) Agar-like polysaccharide produced by a pseudomonas species: production and basic properties. Appl. Environ. Microbiol., 43: 1086-1091.
S.-W. Kim, J.-B. Kim, W.-H. Jung, J.-H. Kim, J.-K. Jung (2006) Over-production of beta-carotene from metabolically engineered Escherichia coli, Biotechnology Letters, 28: 897-904.
K. N. C. Murthy, A. Vanitha, J. Rajesha, M. M. Swamy, P. R. Sowmya, G. A. Ravishankar (2005) In vivo antioxidant activity of carotenoids from Dunaliella salina—a green microalga. Life Sciences, 76: 1381-1390.
A. D. Schmidt, T. Heinekamp, M. Matuschek, B. Liebmann, C. Bollschweiler, A. A. Brakhage (2005) Analysis of mating-dependent transcription of Blakeslea trispora carotenoid biosynthesis genes carB and carRA by quantitative real-time PCR. Applied Microbiology and Biotechnology, 67: 549-555.
A. Schieber, R. Carle (2005) Occurrence of carotenoid cis-isomers in food: Technological, analytical, and nutritional implications. Trends in Food Science & Technology, 16: 416-422.
C. Silva, J. M. S. Cabral, F. van Keulen (2004) Isolation of a β-carotene over-producing soli bacterium, Sphingomonas sp., Biotechnology Letters, 26: 257-262.
K.-J. Yeum, R. M. Russell (2002) Carotenoid bioavailability and bioconversion. Annual Review of Nutrition, 22: 483-504.

1-22. (canceled)

23. A process for the production of high purity carotenoids using a naturally occurring bacterial strain over-producing carotenoids, or a mutant thereof, comprising:
a) inducing a mutation in a selected naturally occurring bacterial strain which over-produces carotenoids or a mutant thereof and screening for a mutant strain with improved total carotenoid accumulation or improved accumulated fraction of single carotenoid in relation to total carotenoids;
b) culturing said bacterial strain in a fermentation step performed as a submerged culture in a controlled bioreactor at a temperature range between 22° C. and 29° C. and a dissolved oxygen concentration below 30% saturation, and
c) optionally extracting and purifying the intracellularly accumulated carotenoid from the obtained biomass.

24. A process, according to claim 23, wherein the selected naturally occurring bacterial strain, isolated from any source in nature, particularly soil, which constitutively produces carotenoids, or a mutant thereof, is preferably a bacteria belonging to the Mycobacterium, Pseudomonas, Dietzia, Flavobacterium, Paracoccus, Rhodococcus, Blastomonas, Sphingomonas, Brevibacterium, Erwinia, Pantoea, Agrobacterium, Paracoccus, Erythrobacter, Xanthobacter, Sphingobacteria, Rhodobacter, Gordonia, Rubrobacter, Arthrobacter, Novosphingobium, Nocardia, Corynebacterium, Streptomyces, Enterobacteriaceae, Thermobifida, Enterobacter, Brevundimonas, Roseiflexus, Sphingopyxis, Aurantimonas, Photobacterium, Robiginitalea, Polaribacter, Tenacibaculum, Parvularcula, Deinococcus, Chloroflexus genera, more preferably bacteria belonging to the Mycobacterium, Pseudomonas, Dietzia, Flavobacterium, Paracoccus, Rhodococcus, Blastomonas, Sphingomonas, Brevibacterium, Erwinia, Pantoea, Agrobacterium, Paracoccus, Erythrobacter, Xanthobacter, Sphingobacteria, Rhodobacter, Gordonia, Rubrobacter, Arthrobacter, Novosphingobium, Nocardia, Corynebacterium, Streptomyces genera, most preferably a bacteria belonging to the Mycobacterium, Pseudomonas, Dietzia, Flavobacterium, Paracoccus, Rhodococcus, Blastomonas, Sphingomonas, Brevibacterium, Erwinia, Pantoea, Paracoccus, Erythrobacter, Xanthobacter, Rhodobacter, Gordonia, Novosphingobium, Nocardia, Corynebacterium genera, of utmost preference a bacteria belonging to the Mycobacterium, Pseudomonas, Dietzia, Flavobacterium, Paracoccus, Rhodococcus, Blastomonas and Sphingomonas genera, particularly a bacteria belonging to the Sphingomonas genus, most preferably a bacteria belonging to the Sphingomonas genus in which the base sequence of DNA corresponding to 16S ribossomal RNA is substantially homologous to the base sequence described in SEQ ID No 1.

25. A process, according to claim 23, wherein the screening for a bacterial strain, comprises selecting mutant strains subjected to the action of at least one mutagenic agent, preferably UV radiation, methanesulfonate or nitrosoguanidine, most preferably methanesulfonate or nitrosoguanidine, with an increase of at least 5% of accumulated total carotenoids or single carotenoid, preferably beta-carotene per unit of biomass or unit of culture liquid when compared to the parent strain or an increase of at least 5% of the accumulated fraction of single carotenoid, preferably beta-carotene, in relation to total carotenoids when compared to the parent strain.

26. A process, according to claim 25, wherein the mutant Sphingomonas strain obtained by the screening is the strain M63Y, with SEQ ID No 2.

27. A process, according to claim 23, comprising growing of the selected bacterial strain in a submerged culture fermentation at a temperature of 24° C.-28° C.

28. A process, according to claim 23, further comprising keeping the oxygen concentration below 10% air saturation, preferably below 5% air saturation, most preferably below 2% air saturation.

29. A process, according to claim 27, wherein the pH of the culture is controlled by means of the addition of acid and/or alkali and/or carbon source preferably within the range of 6.0-8.0, most preferably within the range 6.4-7.6.

30. A process according to claim 23, wherein the extraction and purification of the carotenoids is performed with a mixture of a ketone and an alcohol, most preferably a mixture of acetone and ethanol, most preferably a mixture of acetone and methanol, at a ketone/alcohol ratio of 0/1 to 1/0, preferably at a ketone/alcohol ratio of 1/9 to 9/1, most preferably at a ketone/alcohol ratio of 2/7 to 7/2.

31. A process, according to claim 30, wherein extraction includes a liquid-liquid extraction wherein a hydrophobic solvent or a mixture of hydrophobic solvents hexane and tert-butylmethyl ether, is used as extractant.

32. A process, according to claim 31, comprising a step of carotenoid crystallization.

33. Use of the process according to claim 23 for the production of high purity carotenoids, preferably substantially pure beta-carotene, with a purity grade of an increasing order of preference of 96%, 97%, 98%, 99% or more.

34. High purity carotenoids obtained by the process according to claim 1.

35. A Sphingomonas strain M63Y obtained by the screening method for mutants of claim 25.

36. A Sphingomonas strain M63Y characterized in that it is defined by SEQ ID No 2.

37. A Sphingomonas strain M63Y defined by the following biochemical and growth profile parameters: is Gram-negative, rod shaped and non-spore forming, growing as round, smooth, orange colonies on nutrient agar, between 20 and 30° C., with optimum growth at 27° C., containing meso-diaminopimelic acid (meso-Dpm), typical of the peptidoglycan type A1γ, having ubiquinone-10 has the major isoprenoid quinone and 18:1 w7c the major fatty acid, producing polar lipids, including sphingoglycolipids, and carotenoids, mainly beta-carotene, with a G+C content of the DNA of the strain M63Y was 66.6 mol.

38. Use of Sphingomonas strain M63Y according claim 35 for the production of carotenoids, preferably beta-carotene.

39. Use of Sphingomonas strain M63Y according to claim 36 for the production of carotenoids, preferably beta-carotene.

40. Use of Sphingomonas strain M63Y according to claim 37 for the production of carotenoids, preferably beta-carotene.

41. A process, according to claim 24, wherein the screening for a bacterial strain, comprises selecting mutant strains subjected to the action of at least one mutagenic agent, preferably UV radiation, methanesulfonate or nitrosoguanidine, most preferably methanesulfonate or nitrosoguanidine, with an increase of at least 5% of accumulated total carotenoids or single carotenoid, preferably beta-carotene per unit of biomass or unit of culture liquid when compared to the parent strain or an increase of at least 5% of the accumulated fraction of single carotenoid, preferably beta-carotene, in relation to total carotenoids when compared to the parent strain.

42. A process, according to claim 30, comprising a step of carotenoid crystallization.


SEQUENCE LISTING (2 sequences)

SEQ ID NO: 1 - DNA, 976 bp, Sphingomonas sp.
  misc_feature (965)..(965): n is a, c, g, or t
smcgmgctgacgacagccatgcagcacctgtgttccagtccccgaagaaygagatcggtctcccgaaatcgtccggacatgtcaaacgctggtaaggttctgcgcgttgcttcgaattaaaccacatgctccaccgcttgtgcaggcccccgtcaattcatttgagttttaaccttgcggccgtactccccaggcggataacttaatgcgttastgcgccaccgaagctctaagagccccgacagctagttatcatcgtttacggcgtggactaccagggtatctaatcctgtttgctccccacgctttcgcacctcagcgtcaataccagtccagtgagccgccttcgccactggtgttcttccgaatatctacgaatttcacctctacactcggaattccactcacctctcctggattcaagcgatgcagtcttaaaggcaattctggagttgagctccaggctttcacctctaacttacaaagccgcctacgtgcgctttacgcccagtaattccgaataacgctagctccctccgtattaccgcggctgctggcacggagttagccggagcttattctcccggtactgtcattatcatcccgggtaaaagagctttacaaccctaaggccttcatcactcacgcggcattgctggatcaggctttcgcccattgtccaatattccccactgctgcctcccgtagaggtctggggccgtgtctcagtcccagtgtggctgatcatcctctcagaccagctaaggatcgtcgccttggtgcgcytttacacacaactagctaatctacgcgggctcatcctcggcgataatctttggattacgtskcatcggtatagcatcgttccaatgtkatcgaacaagggcaattccagcgttagcacstggccataagcgaacttcgtcgattgcagtttagcagcgccasttcwttgacaghkmgynynbygaaaaaa

SEQ ID NO: 2 - DNA, 1468 bp, Sphingomonas M63Y
atcctggctcagaatgaacgctggcggcatgcctaacacatgcaagtcgaacgaaggcttcggccttagtggcgcacgggtgcgtaacgcgtgggaatctgccccttggttcggaataacagttggaaacgactgctaataccggatgacgacgtaagtccaaagatttatcgccgagggatgagcccgcgtaggattagctagttggtgtggtaaaggcgcaccaaggcgacgatccttagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttattcggaattactgggcgtaaagcgcacgtaggcggctttgtaagttagaggtgaaagcctggagctcaactccagaattgcctttaagactgcatcgcttgaatccaggagaggtgagtggaattccgagtgtagaggtgaaattcgtagatattcggaagaacaccagtggcgaaggcggctcactggactggtattgacgctgaggagcgaaagcgtggggagcaaacaggattagataccctggtagtccacgccgtaaacgatgataactagctgtcggggctcttagagcttcggtggcgcagctaacgcattaagttatccgcctggggagtacggccgcaaggttaaaactcaaatgaattgacgggggcctgcacaagcggtggagcatgtggtttaattcgaagcaacgcgcagaaccttaccagcgtttgacatgtccggacgatttcgggagaccgatctcttcccttcggggactggaacacaggtgctgcatggctgtcgtcagctcgtgtcgtgagatgttgggttaagtcccgcaacgagcgcaaccctcgcctttagttaccatcattcagttggggactctaaaggaaccgccggtgataagccggaggaaggtggggatgacgtcaagtcctcatggcccttacgcgctgggctacacacgtgctacaatggcggtgacagtgggcagcaatcccgcaagggtgcgctaatctccaaaagccgtctcagttcggattgttctctgcaactcgagagcatgaaggcggaatcgctagtaatcgcggatcagcatgccgcggtgaatacgttcccaggccttgtacacaccgcccgtcacaccatgggagttggattcacccgaaggcagtgcgctaaccgcaaggaggcagctgaccacggtgggttcagcgactggggtgaagtcgtaacaaggtagccgtaggggaacctgcggctggatcacctcct